(19) United States
(12) Patent Application Publication
(10) Pub. No.: US 2026/0093872 A1
(43) Pub. Date: Apr. 2, 2026

(54) CONFORMER GENERATION USING ROTATION-AVERAGED FLOW-MATCHING IN GENERATIVE ARTIFICIAL INTELLIGENCE MODELS

(71) Applicant: NVIDIA Corporation, Santa Clara, CA (US)

(72) Inventors: Mario GEIGER, Lausanne (CH); Zhonglin CAO, San Jose, CA (US); Emine KUCUKBENLI, Melrose, MA (US)

(21) Appl. No.: 19/235,342

(22) Filed: Jun. 11, 2025

Related U.S. Application Data
(60) Provisional application No. 63/701,087, filed on Sep. 30, 2024.

(51) Int. Cl. G06F 30/27; G16C 10/00; G16C 20/70

(52) U.S. Cl. CPC G06F 30/27; G16C 10/00; G16C 20/70

(57) ABSTRACT

In various examples, methods for training generative artificial intelligence models to generate conformers based on an average flow loss over a universe of rotations of a conformer include modeling a flow objective over a universe of conformers for a molecule having a defined set of atoms and bonds between atoms, the flow objective being modeled based on an integration over a universe of ground-truth conformer rotations; calculating an average flow loss based on a difference between the modeled flow objective and a ground-truth flow objective associated with the integration over the universe of ground-truth conformer rotations; training a generative model to generate a conformer given an input of atoms and bonds of a target molecule, the training being based at least on minimizing the average flow loss; and deploying the trained generative model.

### CROSS-REFERENCE TO RELATED APPLICATIONS

This application claims priority to and benefit of U.S. Provisional Patent Application Ser. No. 63/701,087, entitled “Conformer Generation,” filed Sep. 30, 2024, and assigned to the assignee hereof, the entire contents of which are hereby incorporated by reference.

### TECHNICAL FIELD

Embodiments of the present disclosure relate generally to conformer generation using generative artificial intelligence models, and more specifically to techniques for improving the computational efficiency of generating conformers using generative artificial intelligence models.

### BACKGROUND

Molecules are structures defined by molecular bonds between different atoms in a three-dimensional space. An atom within a molecule may have any number of bonds with other molecules with bond lengths (defined in angstroms (Å)) and bond angles between adjacent bonds. In the molecular virtual screening and design tasks, generating these three-dimensional molecular structures may allow for molecules to be evaluated for their potential as a therapeutic (e.g., a small molecule therapeutic, a large molecule therapeutic (e.g., a biologic product), another protein, etc.). For example, based on three-dimensional molecules generated in the process of virtual screening or drug discovery, affinity predictions between a protein (with an a priori known structure) and a ligand (e.g., a generated three-dimensional molecule that is designed to bond to the protein) can be predicted to determine whether a molecule is a candidate for synthesis and testing for a therapeutic effect relative to the target molecule. For example, molecules with high predicted binding affinities may be molecules with a strong bond in a biological complex (e.g., molecules that are ionically bonded, molecules that are bonded based on a large number of shared electrons in a covalent bond, etc.) and thus may be candidates for synthesis and testing. Meanwhile, molecules with low predicted binding affinities may have weak bonds in a biological complex (e.g., molecules that are bonded by hydrogen bonds, molecules bonded by van der Waals interaction, etc.) and thus may not be candidates for synthesis and testing.

A molecule may have any number of conformers, or three-dimensional arrangements of atoms in the molecule given a two-dimensional graph representation of the molecule. The validity of a conformer may be a prerequisite for accurately performing various computational chemistry tasks, such as structure-based and ligand-based virtual screening tasks for determining whether a conformer or a molecule is a potential therapeutic for a given disease. However, generating valid conformers using molecular dynamics simulations may be computationally expensive. Meanwhile, rule-based techniques may be less computationally expensive than molecular dynamics simulation techniques but may not result in the generation of accurate conformers or may not generate conformers at some local minima in a potential energy space.

As the foregoing illustrates, what is needed in the art are more effective techniques for computational generation of conformers.

### BRIEF DESCRIPTION OF DRAWINGS

FIG. 1 illustrates a block diagram of a computing system configured to implement one or more aspects of at least one embodiment;

FIG. 2 illustrates a pipeline for generating a conformer using a generative artificial intelligence model trained based on rotation-averaged flow matching, according to at least one embodiment;

FIG. 3 illustrates example operations for training a generative artificial intelligence model to generate conformers based on rotation-averaged flow matching;

FIG. 4 illustrates example operations for generating conformers using a generative artificial intelligence model trained based on rotation-averaged flow matching;

FIG. 5A illustrates inference and/or training logic, according to at least one embodiment;

FIG. 5B illustrates inference and/or training logic, according to at least one embodiment; and

FIG. 6 illustrates training and deployment of a neural network, according to at least one embodiment.

### DETAILED DESCRIPTION

As discussed herein, identification of conformers for testing as potential treatments for various medical conditions is an important task in drug discovery. By identifying conformers for testing, the process of drug discovery may be accelerated, and resources may be dedicated to testing molecules that are more likely to have a therapeutic effect than those that are less likely to have a therapeutic effect or are likely to have no therapeutic effect at all. As discussed above, however, generating conformers may be computationally expensive, and less computationally expensive techniques such as rule-based tools may not generate accurate conformers or conformers at local minima in a potential energy surface.

Molecular conformer generation tasks generally involve the sampling of a conformer from an intractable conformer distribution conditioned on a two-dimensional graph representation of a conformer. Because a conformer may have a variety of structures and any rotation of a structure may be a valid rotation, generating a conformer may be a computationally expensive task. For example, generative models may be trained to generate a conformer, and the generation process may be stochastic due to the properties of conformers. Given a set of atoms in a molecule and a set of bonds between the atoms in the molecules, molecular conformer generation may be based on random changes to the positions of atoms in a molecule in a Euclidean space that results in a structure that conforms to various rules defining the structure of a molecule (e.g., bond strength and distance, bond type, etc.). Regression models may also be used to generate conformers based on operations performed on substructures of a molecule (e.g., subsets of atoms and bonds in an overall molecule). Generally, however, techniques for generating conformers using generative artificial intelligence models involve a computational expense (e.g., processor utilization, memory utilization, bandwidth, etc.) that scales infeasibly as the number of molecules to be generated and examined (e.g., in computational simulation tasks for drug discovery, screening, etc.) increases.

To compensate for the computational expense involved in conformer generation using generative artificial intelligence models, embodiments described herein use an averaged flow metric calculated over a universe of rotations for conformers to train a generative artificial intelligence model to generate conformers from a two-dimensional graph representation of a molecule. Because any rotation in the Euclidean space of a conformer is a valid conformer, training a generative artificial intelligence model using flow matching techniques based on average flow over the universe of rotations for a conformer (e.g., all possible ways a conformer can be rotated in three-dimensional space) generally reduces the computational expense involved in training a generative model for generating conformers from a two-dimensional graph of a molecule. For example, by using an average flow metric to train a generative artificial intelligence model, data distributions used in training the generative model need not be rotationally aligned, as the averaged path from a sampled data distribution (e.g., of coordinates in a Euclidean space for an atom in a molecule) to a ground-truth data distribution may cover a flow over the universe of rotations. Thus, the training of a generative artificial intelligence model using an averaged flow metric calculated over a universe of rotations may accelerate convergence to a training objective. Further, embodiments presented herein may improve the quality and accuracy of conformers generated by a generative artificial intelligence model.

The above examples are not in any way intended to be limiting. As persons skilled in the art will appreciate, as a general matter, the techniques for automatically generating dialogue flows from unlabeled conversation data can be implemented in any suitable application.

The systems and methods described herein may be used for a variety of purposes, by way of example and without limitation, for use in systems associated with machine control, machine locomotion, machine driving, synthetic data generation, model training, perception, augmented reality, virtual reality, mixed reality, robotics, security and surveillance, simulation and digital twinning, autonomous or semi-autonomous machine applications, deep learning, environment simulation, data center processing, conversational AI, generative AI, light transport simulation (e.g., ray-tracing, path tracing, etc.), collaborative content creation for 3D assets, cloud computing and/or any other suitable applications.

Disclosed embodiments may be comprised in a variety of different systems such as automotive systems (e.g., an infotainment or plug-in gaming/streaming system of an autonomous or semi-autonomous machine), systems implemented using a robot, aerial systems, medial systems, boating systems, smart area monitoring systems, systems for performing deep learning operations, systems for performing simulation operations, systems for performing digital twin operations, systems implemented using an edge device, systems incorporating one or more virtual machines (VMs), systems for performing synthetic data generation operations, systems implemented at least partially in a data center, systems for performing conversational AI operations, systems implementing one or more language models-such as large language models (LLMs), small language models (SLMs), vision language models (VLMs), and/or multi-modal language models that may process text, audio, and/or image data, systems for performing light transport simulation, systems for performing collaborative content creation for 3D assets (e.g., systems or platforms that use universal scene descriptor (USD) data, such as OpenUSD), systems implemented at least partially using cloud computing resources, systems for performing generative AI operations, and/or other types of systems.

### System Overview

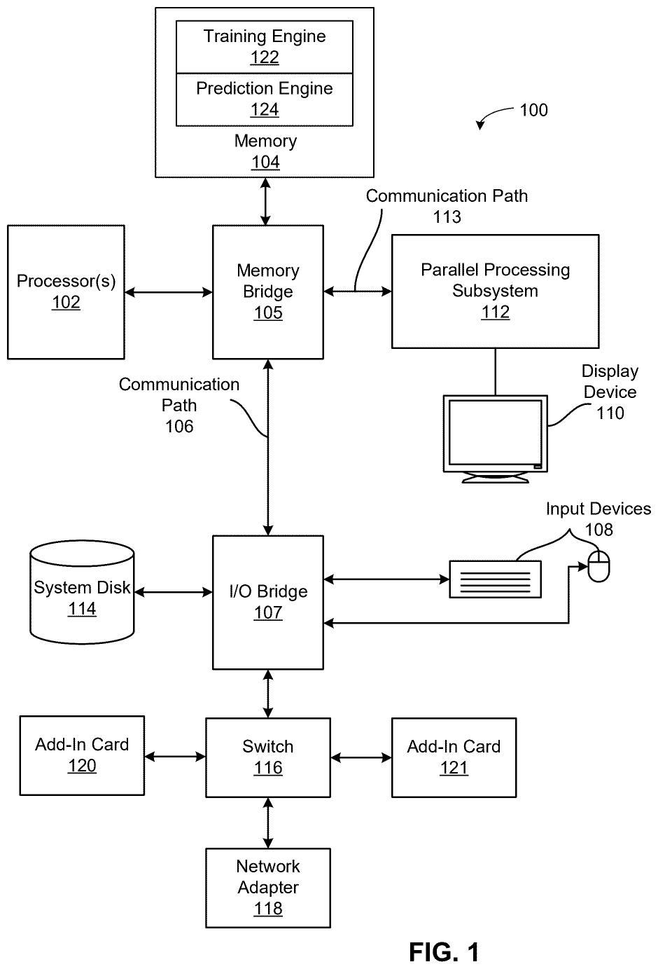

FIG. 1 is a block diagram illustrating a computing system 100 configured to implement one or more aspects of at least one embodiment. In at least one embodiment, computing system 100 may include any type of computing device, including, without limitation, a server machine, a server platform, a desktop machine, a laptop machine, a hand-held/mobile device, a digital kiosk, an in-vehicle infotainment system, a smart speaker or display, a television, and/or a wearable device. In at least one embodiment, computing system 100 is a server machine operating in a data center or a cloud computing environment that provides scalable computing resources as a service over a network.

In various embodiments, computing system 100 includes, without limitation, one or more processors 102 and one or more memories 104 coupled to a parallel processing subsystem 112 via a memory bridge 105 and a communication path 113. Memory bridge 105 is further coupled to an I/O (input/output) bridge 107 via a communication path 106, and I/O bridge 107 is, in turn, coupled to a switch 116.

In one embodiment, I/O bridge 107 is configured to receive user input information from optional input devices 108, such as (but not limited to) a keyboard, mouse, touch screen, sensor data analysis (e.g., evaluating gestures, speech, or other information about one or more uses in a field of view or sensory field of one or more sensors), a VR/MR/AR headset, a gesture recognition system, a steering wheel, mechanical, digital, or touch sensitive buttons or input components, and/or a microphone, and forward the input information to processor(s) 102 for processing. In at least one embodiment, computing system 100 may be a server machine in a cloud computing environment. In such embodiments, computing system 100 may omit input devices 108 and receive equivalent input information as commands (e.g., responsive to one or more inputs from a remote computing device) and/or messages transmitted over a network and received via the network adapter 118. In at least one embodiment, switch 116 is configured to provide connections between I/O bridge 107 and other components of computing system 100, such as a network adapter 118 and various add-in cards 120 and 121.

In at least one embodiment, I/O bridge 107 is coupled to a system disk 114 that may be configured to store content and applications and data for use by processor(s) 102 and parallel processing subsystem 112. In one embodiment, system disk 114 provides non-volatile storage for applications and data and may include fixed or removable hard disk drives, flash memory devices, and CD-ROM (compact disc read-only-memory), DVD-ROM (digital versatile disc-ROM), Blu-ray, HD-DVD (high-definition DVD), or other magnetic, optical, or solid state storage devices. In various embodiments, other components, such as universal serial bus or other port connections, compact disc drives, digital versatile disc drives, film recording devices, and the like, may be connected to I/O bridge 107 as well.

In various embodiments, memory bridge 105 may be a Northbridge chip, and I/O bridge 107 may be a Southbridge chip. In addition, communication paths 106 and 113, as well as other communication paths within computing system 100, may be implemented using any technically suitable protocols, including, without limitation, AGP (Accelerated Graphics Port), HyperTransport, or any other bus or point-to-point communication protocol known in the art.

In at least one embodiment, parallel processing subsystem 112 includes a graphics subsystem that delivers pixels to an optional display device 110 that may be any conventional cathode ray tube, liquid crystal display, light-emitting diode display, and/or the like. In such embodiments, parallel processing subsystem 112 may incorporate circuitry optimized for graphics and video processing, including, for example, video output circuitry. Such circuitry may be incorporated across one or more parallel processing units (PPUs), also referred to herein as parallel processors, included within the parallel processing subsystem 112.

In at least one embodiment, parallel processing subsystem 112 incorporates circuitry optimized (e.g., that undergoes optimization) for general purpose and/or compute processing. Again, such circuitry may be incorporated across one or more PPUs included within parallel processing subsystem 112 that are configured to perform such general purpose and/or compute operations. In yet other embodiments, the one or more PPUs included within parallel processing subsystem 112 may be configured to perform graphics processing, general purpose processing, and/or compute processing operations. Memor(ies) 104 include at least one device driver configured to manage the processing operations of the one or more PPUs within parallel processing subsystem 112. In addition, memor(ies) 104 include instructions implementing a training engine 122 and a prediction engine 124, which can be executed by processor(s) and/or parallel processing subsystem 112.

In various embodiments, parallel processing subsystem 112 may be integrated with one or more of the other elements of FIG. 1 to form a single system. For example, parallel processing subsystem 112 may be integrated with processor(s) 102 and other connection circuitry on a single chip to form a system on a chip (SoC).

Processor(s) 102 may include any suitable processor implemented as a central processing unit (CPU), a graphics processing unit (GPU), an application-specific integrated circuit (ASIC), a field programmable gate array (FPGA), an artificial intelligence (AI) accelerator, a deep learning accelerator (DLA), a parallel processing unit (PPU), a data processing unit (DPU), a vector or vision processing unit (VPU), a programmable vision accelerator (PVA) (which may include one or more VPUs, pixel processing engines (PPEs), and/or direct memory access (DMA) systems), any other type of processing unit, or a combination of different processing units, such as a CPU(s) configured to operate in conjunction with a GPU(s). In general, processor(s) 102 may include any technically feasible hardware unit capable of processing data and/or executing software applications. Further, in the context of this disclosure, the computing elements shown in computing system 100 may correspond to a physical computing system (e.g., a system in a data center or a machine) and/or may correspond to a virtual computing instance executing within a computing cloud.

In at least one embodiment, processor(s) 102 issue commands that control the operation of PPUs. In at least one embodiment, communication path 113 is a Peripheral Component Interconnect Express (PCIe) link, in which dedicated lanes are allocated to each PPU. Other communication paths may also be used. The PPU advantageously implements a highly parallel processing architecture, and the PPU may be provided with any amount of local parallel processing memory (PP memory).

It will be appreciated that the system shown herein is illustrative and that variations and modifications are possible. The connection topology, including the number and arrangement of bridges, the number of processors 102, and the number of parallel processing subsystems 112, may be modified as desired. For example, in at least one embodiment, memor(ies) 104 may be connected to processor(s) 102 directly rather than through memory bridge 105, and other devices may communicate with memor(ies) 104 via memory bridge 105 and processors 102. In other embodiments, parallel processing subsystem 112 may be connected to I/O bridge 107 or directly to processor(s) 102, rather than to memory bridge 105. In still other embodiments, I/O bridge 107 and memory bridge 105 may be integrated into a single chip instead of existing as one or more discrete devices. In certain embodiments, one or more components shown in FIG. 1 may not be present. For example, switch 116 may be eliminated, and network adapter 118 and add-in cards 120, 121 would connect directly to I/O bridge 107. Further, in certain embodiments, one or more components shown in FIG. 1 may be implemented as virtualized resources in a virtual computing environment, such as a cloud computing environment. In particular, the parallel processing subsystem 112 may be implemented as a virtualized parallel processing subsystem in at least one embodiment. For example, the parallel processing subsystem 112 may be implemented as a virtual graphics processing unit(s) (vGPU(s)) that renders graphics on a virtual machine(s) (VM(s)) executing on a server machine(s) whose GPU(s) and other physical resources are shared across one or more VMs.

In some embodiments, training engine 122 and a prediction engine 124 include functionality to train and use machine learning models to generate conformers from an input of a two-dimensional graph representation of a molecule. As discussed in further detail herein, a generative artificial intelligence model for generating conformers from a two-dimensional graph representation of a molecule may be trained based on an averaged flow objective. The averaged flow objective generally allows for a model to be trained based on the universe of possible rotations of a conformer using a single flow objective instead of flow objectives over each possible rotation of a conformer. By doing so, as discussed, embodiments presented herein may train and use models with increased computational efficiency than techniques that train a model based on rotations of data distributions. Further, the resulting generative model trained using the techniques discussed herein may generate conformers with increased quality and diversity and decreased computational expense than cheminformatics models, rule-based models, and generative models trained using other techniques.

### Generative Artificial Intelligence Models for Generating Conformers Based on Averaged Flow Over Rotations of a Conformer

Generally, an averaged flow metric used in training a generative artificial intelligence model models a probability density path pₜ(xₜ) that transforms a noise distribution at t=0 to a data distribution at t=1. In doing so, the path pₜ(xₜ) may correspond to a flow ψₜ that pushes samples from an initial probability distribution p₀ to a target probability distribution pₜ via pₜ=[ψ]ₜ*p₀, where * denotes a push-forward function. The flow ψₜ may be modeled via the ordinary differential equation

$${dx}_{t} = {v}_{t}^{θ} ( {x}_{t} ) dt ,$$

in which

$${v}_{t}^{θ} ( {x}_{t} )$$

is a learnable vector field with parameters θ. From initialization from noise x₀˜p₀(x₀), the differential equation

$${dx}_{t} = {v}_{t}^{θ} ( {x}_{t} ) dt$$

simulates the flow and transforms noise into an approximate data distribution sample. The probability density path pₜ(xₜ) corresponding to the flow ψₜ is related to a ground-truth vector field uₜ(xₜ) via the continuity equation dpₜ(x)/dt=−∇ₓ·(pₜ(x)uₜ(x)). The ground-truth vector field uₜ(xₜ) may be an intractable vector field.

To construct the probability density path pₜ, a conditional probability pₜ(x|xₜ) and a conditional vector field uₜ(x|x₁) may be constructed as:

$${p}_{t} ( x ) = \int {p}_{t} ( x \vert {x}_{1} ) q ( {x}_{1} ) {dx}_{1}$$

$$and$$

$${u}_{t} ( x ) = \int {u}_{t} ( x \vert {x}_{1} ) \frac{{p}_{t} ( x \vert {x}_{1} ) q ( {x}_{1} )}{{p}_{t} ( x )} {dx}_{1}$$

where x₁ corresponds to the target data distribution and uₜ(x) represents a vector that transforms a noise distribution or other data distribution to a target data distribution. In the context of rotation-invariant conformers, the target data distribution may be conformer shapes distributed across a universe of valid shapes, each of which may be valid for any rotation of a conformer in the Euclidean space).

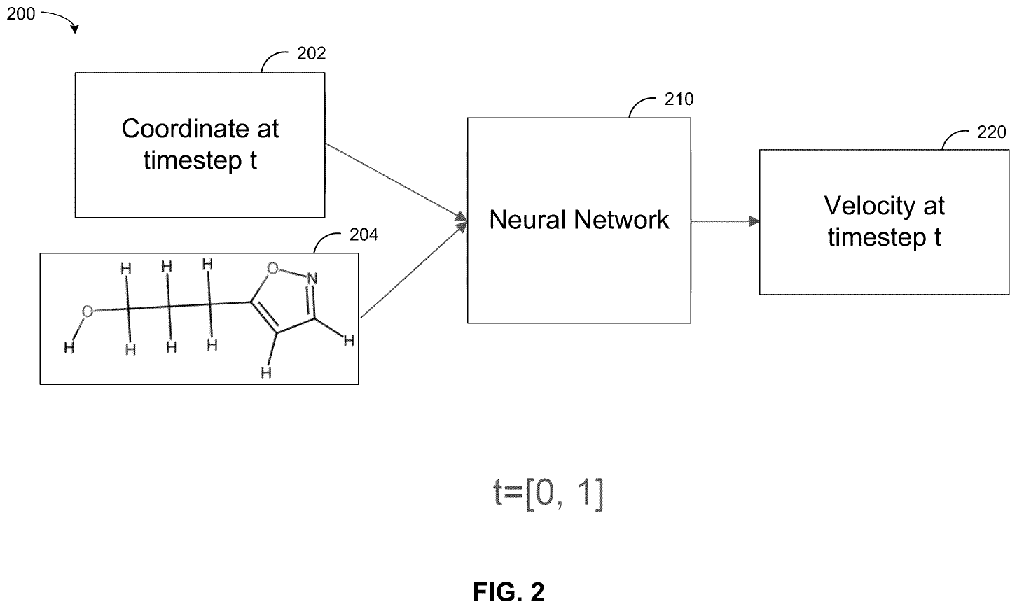

FIG. 2 illustrates a pipeline 200 for generating a conformer, according to at least one embodiment. In some embodiments, the pipeline 200 may be trained by the training engine 122 illustrated in FIG. 1, and inferencing operations for generating conformers using a trained neural network 210 may be executed by the prediction engine 124 illustrated in FIG. 1.

Training engine 122 generally trains the neural network 210 based on the observation that a data distribution q covering a target molecule may exhibit group symmetries that can be integrated out. That is, the data distribution q may include transformations, such as rotations, that leave the underlying distribution changed. In the context of conformers, because a rotation along any combination of axes of a conformer results in a post-rotation conformer having the same structure as a pre-rotation conformer, a conformer can be considered a symmetry group G including transformations g:x [symbol] g·x that leave the data distribution q unchanged. For a molecule that can be represented as a Lie group with a Haar measure, the data distribution q can be expressed according to the equation:

$$q ( x ) = \int d \hat{x} \hat{q} ( \hat{x} ) \int dg {δ}_{g \cdot \hat{x}} ( x )$$

where \hat{q} represents the distribution over the group orbits, \hat{x} is a representative point of the orbit, and the integral over G uses the Haar measure.

Based on the definition of the data distribution q, the flow objective uₜ may be rewritten to be rotation-invariant according to the equation:

$${u}_{t} ( x ) = \int d \hat{x} \hat{q} ( \hat{x} ) \int dg {u}_{t} ( x \vert g \cdot \hat{x} ) \frac{{p}_{t} ( x \vert g \cdot \hat{x} )}{{p}_{t} ( x )}$$

where pₜ(x)=∫d\hat{x}\hat{q}(\hat{x})∫dg pₜ(x|g·\hat{x}) is a partition function.

For generating a conformer, x may be an N×3 matrix representing the 3D coordinates of the N atoms in the conformer, and G may be defined as a rotation group SO(3) for a conformer. A rotation matrix R for the symmetry group G may act on x as x [symbol] xR^{T} (that is, may modify x based on a transpose of the rotation matrix R). To train the neural network 210 to generate molecular conformers that conform to local minima in a potential energy surface (or other conformational energy landscape), the orbits \hat{x} may correspond to different low-energy conformers in a molecule and the permutations that allow an input two-dimensional molecular graph to remain invariant. Thus, the integral ∫d\hat{x} \hat{q}(\hat{x}), representing an integral over an ensemble of conformers, can be written as Σ_{\hat{x}∈conformers} \hat{q}(\hat{x}), where \hat{q}(\hat{x}) is a weight associated with a conformer.

The path pₜ(x|x₁), or the probability density path to x conditioned on x₁, may be a Gaussian distribution defined by the expression:

$${p}_{t} ( x \vert {x}_{1} ) ∝ exp ( \frac{1}{2} \frac{1}{{( 1 - t )}^{2}} {\sum }_{ij δ} {( x - {tx}_{1} )}_{i δ} {\sum }_{ij} {( x - {tx}_{1} )}_{j δ} ) ≡ exp ( \frac{1}{2} \frac{{‖ x - {tx}_{1} ‖}_{Σ}^{2}}{{( 1 - t )}^{2}} )$$

where Σ is a [symbol] ^{N×N} matrix, i and j are atom indices, and δ is an index of the coordinates (e.g., in a Cartesian space) at which atoms are located.

Based on the above, the ground-truth vector field uₜ(x) may be rewritten according to the equation:

$${u}_{t} ( x ) = \frac{1}{{Z}_{t} ( x , 0 )} {\sum }_{\hat{x} ∈ conformers} \hat{q} ( \hat{x} ) {\int }_{SO ( 3 )} dR \frac{\hat{x} {R}^{T} - x}{1 - t} {e}^{- \frac{1}{2} \frac{{‖ x - t \hat{x} {R}^{T} ‖}_{Σ}^{2}}{{( 1 - t )}^{2}}}$$

Zₜ represents a normalization factor over the universe of conformers, defined by the equation:

$${Z}_{t} ( x , α ) = {\sum }_{\hat{x} ∈ conformers} \hat{q} ( \hat{x} ) {\int }_{SO ( 3 )} dR {e}^{- \frac{1}{2} \frac{{‖ x - t \hat{x} {R}^{T} ‖}_{Σ}^{2}}{{( 1 - t )}^{2}} + tr ( {α}^{T} \hat{x} {R}^{T} )}$$

where α is an N×3 matrix.

The ground-truth vector field uₜ(x) can be computed based on the derivative of log Zₜ(x, α) with respect to a and evaluating the derivative of log Zₜ(x, α) at α=0. log Zₜ(x, α) may be expressed according to the equation:

$$log {Z}_{t} ( x , α ) = log {\sum }_{\hat{x} ∈ conformers} \hat{q} ( \hat{x} ) exp ( log {\int }_{SO ( 3 )} dR exp tr ( ( {α}^{T} + \frac{t}{{( 1 - t )}^{2}} {x}^{T} \sum ) \hat{x} {R}^{T} ) + constant in α )$$

The integral over R, the universe of rotations for the conformer, may be solved using a closed-form solution based on the equation:

$$F = {α}^{T} \hat{x} + \frac{t}{{( 1 - t )}^{2}} {x}^{T} \sum \hat{x}$$

where F represents any arbitrary 3×3 matrix.

Based on solving the logarithm of the normalization factor Zₜ over the universe of conformers, the training engine 122 can define the ground-truth vector field uₜ(xₜ) according to the equation:

$${u}_{t} ( {x}_{t} ) = \frac{{[ {\partial }_{α} log {Z}_{t} ( {x}_{t} , α ) ]}_{α = 0} - {x}_{t}}{1 - t}$$

where xₜ represents an intermediate particle.

Subsequently, the training engine 122 can train the neural network 210 based on directly learning an average flow loss, [symbol] _{AvgFlow}. The average flow loss may be calculated within the Euclidean space based on a difference between a learnable vector field

$${v}_{t}^{θ} ( {x}_{t} )$$

and the ground-truth vector field uₜ(xₜ), according to the equation:

$${ℒ}_{AvgFlow} ( θ ) = 𝔼 [ {‖ {v}_{t}^{θ} ( {x}_{t} ) - {u}_{t} ( {x}_{t} ) ‖}^{2} ] with t ∈ [ 0 , 1 ]$$

As discussed, by training a model to generate a conformer based on an average flow loss [symbol] _{AvgFlow}, which is a loss calculated over a universe of rotations of a conformer, the training engine 122 can more efficiently train a model to generate accurate conformers that comply with quantum chemical rules for atomic binding and are located at local minima in the potential energy surface for a molecule. Training engine 122 can train the neural network 210 using fewer computing resources than would be used in cases in which the neural network 210 is trained using rotational alignments of atomic location distributions for different conformers of a molecule.

A neural network 210 trained using the average flow loss metric discussed above generally allows for a trajectory to be modeled from a noise distribution to the position of an atom in a conformer. The trajectory may not be a direct trajectory, and thus, the neural network 210 may involve multiple iterations (e.g., hundreds or even thousands) of solving differential equations. To further address the computational expense involved in generating conformers from a two-dimensional graph representation of a molecule, the training engine 122 may further refine the neural network 210 based on a reflow loss [symbol] _{Reflow} and/or a distillation loss [symbol] _{Distill}. These refinements generally address the computational expense of sampling conformers using a neural network 210 may straightening a trajectory from an origin point in a noise distribution to the location of an atom in a conformer in the Euclidean space.

To refine the neural network 210 using a reflow loss, the training engine 122 can randomly sample atom coordinates

$${X}_{0}^{′}$$

from a noise distribution (e.g., a Gaussian distribution) and generate a corresponding conformer

$${X}_{1}^{′} .$$

The pairing of

$$( {X}_{0}^{′} , {X}_{1}^{′} )$$

may be used as a rectified flow objective. Based on this pairing, the training engine 122 can finetune the neural network 210 according to the loss term:

$${ℒ}_{Reflow} ( θ ) = 𝔼 [ {‖ {v}_{t}^{θ} ( {X}_{t}^{′} , t ) - ( {X}_{1}^{′} - {X}_{0}^{′} ) ‖}^{2} ] with t ∈ [ 0 , 1 ]$$

Because the pairing of

$$( {X}_{0}^{′} , {X}_{1}^{′} )$$

has an equal or lower transport cost than (X₀, X₁) when X₀ is sampled from a noise distribution and X₁ is sampled from a data distribution, refining the neural network 210 using a reflow loss can reduce the transport cost in moving from a noise distribution to a data distribution and straighten the trajectory in the ground-truth vector field.

In some embodiments, because the trajectory between a noise distribution and a conformer data distribution has higher curvature when t is closer to 0, training engine 122 may sample t from an exponential distribution with a probability density function defined according to the equation:

$$p ( t ) ∝ Exp ( λ t )$$

where λ is selected so that training iterations are focused on t<0.5. By doing so, more training epochs may be performed in areas in which the trajectory is curved, and fewer training epochs may be performed in which the trajectory remains substantially straight. Thus, by focusing the training iterations on t<0.5, training engine 122 can learn the curved portion of a path pₜ(xₜ) with higher fidelity (e.g., more samples) than the straight portion of the path (which may not significantly change in direction in the way a curved portion of the path does).

In some embodiments, training engine 122 may further refine the neural network 210 based on distillation of a relationship between the pairing of

$$( {X}_{0}^{′} , {X}_{1}^{′} ) .$$

By distilling the relationship between

$$( {X}_{0}^{′} , {X}_{1}^{′} ) ,$$

training engine 122 may allow the neural network 210 to perform a single-step transport from data in a Gaussian distribution to a ground-truth data distribution (e.g., of conformers that are SO(3)). During distillation, thus, training engine 122 can fine-tune the neural network 210 based on a distillation loss, [symbol] _{Distill}, defined according to the equation:

$${ℒ}_{Distill} ( θ ) = 𝔼 [ {‖ {v}_{t}^{θ} ( {X}_{0}^{′} , 0 ) - ( {X}_{1}^{′} - {X}_{0}^{′} ) ‖}^{2} ]$$

Prediction engine 124 executes pipeline 200 to generate conformers in response to a received request to generate a conformer. As illustrated, the request generally includes a set of coordinates 202 and a two-dimensional molecular structure 204 defining the molecule for which a conformer is to be generated. The set of coordinates at timestep t=0 may be coordinates in a Gaussian or other noise distribution, while the set of coordinates at other timesteps t∈(0,1) may be coordinates determined based on a vector field generated in a previous inferencing round performed by the prediction engine 124 using neural network 210. The two-dimensional molecular structure 204 generally defines the atoms included in a molecule, bonds between atoms in the molecule, and types of bonds between different atoms (e.g., single bonds, double bonds, etc.).

Neural network 210, as discussed above, is generally trained to generate a conformer based on the input of set of coordinates 202 and the two-dimensional molecular structure 204. To do so, neural network 210 generates a velocity output 220 at a timestep t∈[0,1]. The velocity output generally is associated with a learned vector field

$${v}_{t}^{θ} ( {x}_{t} )$$

that illustrates a direction and magnitude of movement for atoms at different points in a Euclidean space. The output of the neural network 210 at timestep t may be the sum of the input set of coordinates 202 (which, as discussed, may be a noise distribution at timestep t=0 or a denoised set of coordinates generated at timestep t−1) and the vector field

$${v}_{t}^{θ}$$

generated by the neural network 210 at timestep t.

In some embodiments, the neural network 210 may be an equivariant graph neural network. In the equivariant graph neural network, the neural network may be equivariant to rotations, translations, reflections, and/or permutations. Because the neural network 210 may be rotation equivariant, the neural network 210 may generate a valid conformer (e.g., a molecular structure that conforms to bond distance, bond type, and other quantum chemistry rules, a molecular structure having an energy at or near a local minimum in a potential energy surface defined for the molecule, etc.) in any rotation, as any rotation of a conformer may be a valid conformer.

In some embodiments, the neural network 210 may include an interaction block in which atomic features, such as type and position information, are mixed with relative distance vectors identifying a direction and magnitude by which each atom is to move. The interaction block may, for example, include a convolutional block that uses an embedding of the distance vectors edges between atoms in the molecule to generate a new set of positions for atoms in a molecule.

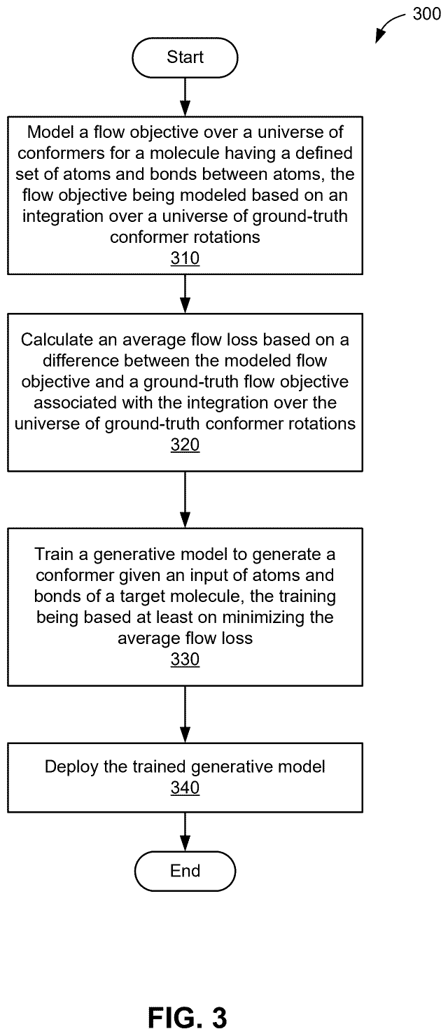

FIG. 3 illustrates example operations 300 for training a model to generate conformers from an input of a two-dimensional graph representation of a molecule based on an averaged flow loss over a universe of rotations for the conformer. The operations 300 may be performed, for example, by a computing device on which a machine learning model can be trained, such as a computing device on which training engine 122 illustrated in FIG. 1 executes. The computing device may be a cloud computing instance, a physical cluster of computers, a server, or the like.

As illustrated, operations 300 begin at block 310, where training engine 122 models a flow objective over a universe of conformers for a molecule having a defined set of atoms and bonds between atoms, the flow objective being modeled based on an integration over a universe of ground-truth conformer rotations.

In some embodiments, the integration over the universe of ground-truth conformer rotations is calculated based on an average atom location over the universe of rotations for an atom in the molecule.

In some embodiments, the modeled flow objective comprises a learned vector field,

$${v}_{t}^{θ} ,$$

associated with a generated conformer. In some embodiments, the ground-truth flow objective comprises a ground-truth vector field, uₜ, associated with a ground-truth conformer.

In some embodiments, the ground-truth vector field comprises a vector field calculated based on a normalized summation of integrals over each rotation of the ground-truth conformer in the universe of ground-truth conformer rotations. The normalization factor, Z, may be defined as a summation over integrals of rotations in the universe of ground-truth conformer rotations. In some embodiments, the normalized summation of integrals over each rotation of the ground-truth conformer in the universe of ground-truth conformer rotations is calculated based on a partial derivative of a partition function over the universe of ground-truth conformer rotations and a location of an intermediate particle.

In some embodiments, the universe of ground-truth conformer rotations comprise an ensemble of conformers for a molecule, each conformer in the ensemble of conformers corresponding to the molecule at a local minimum in a conformational energy landscape.

At block 320, training engine 122 calculates an average flow loss based on a difference between the modeled flow objective and a ground-truth flow objective associated with the integration over the universe of ground-truth conformer rotations.

At block 330, training engine 122 trains a generative model to generate a conformer given an input of atoms and bonds of a target molecule, the training being based at least on minimizing the average flow loss.

At block 340, training engine 122 deploys the trained generative model.

In some embodiments, operations 300 further include the training engine 122 refining the generative model based on a reflow loss calculated based on a position of an atom in the molecule sampled from a noise distribution and a position of the atom in a denoised version of the molecule generated by the generative model. As discussed, the reflow loss may rectify the flow objective to finetune the model and straighten a trajectory from a point in the noise distribution (e.g., a coordinate sample from a Gaussian noise distribution) to a point in a data distribution associated with a ground-truth conformer. In some embodiments, reflow loss may be calculated for samples generated by the generative artificial intelligence model in earlier timesteps in the process of generating a conformer (e.g., in timesteps where t<0.5). By doing so, as discussed, the sampling may allow for higher fidelity in generating vector fields when t is closer to 0 and when the trajectories from a noise distribution to a data distribution have more significant curvature than the straighter trajectories that are observed when t is closer to 1.

In some embodiments, operations 300 further include the training engine 122 refining the generative model based on a distillation loss based on a relationship between a position of an atom in the molecule sampled from a noise distribution and a position of the atom in a denoised version of the molecule generated by the generative model. In some embodiments, training engine 122 refines the generative model based on the distillation loss after refining the generative model based on a reflow loss discussed above. Generally, refinement of the generative model based on the distillation loss may allow for one-step transport from the noise distribution to the data distribution associated with a ground-truth conformer.

In some embodiments, the generative model is trained to generate the conformer based on a direct line from an initial point and a fixed point in a single time step.

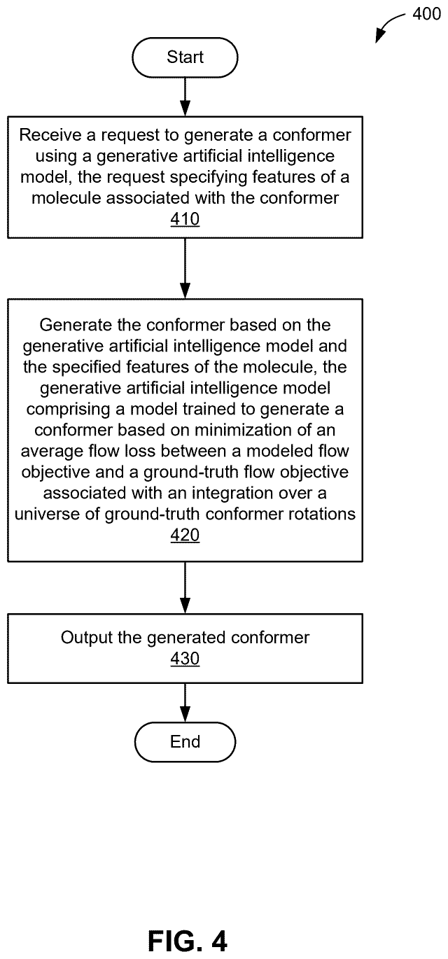

FIG. 4 illustrates example operations 400 for generating conformers from an input of a two-dimensional graph representation of a molecule based on a machine learning model trained based on an average flow loss over a universe of rotations of a conformer. The operations 400 may be performed, for example, by a computing device, such as a desktop computer, a laptop computer, a server, a cloud computing instance, or the like, on which a prediction engine, such as prediction engine 124 illustrated in FIG. 1, executes.

As illustrated, the operations 400 begin at block 410 with prediction engine 124 receiving a request to generate a conformer using a generative artificial intelligence model, the request specifying features of a molecule associated with the conformer.

In some embodiments, the features of the molecule associated with the conformer comprise atom type, bonds between atoms in the molecule, and a type of each bond between atoms in the molecule.

At block 420, prediction engine 124 generates the conformer based on the generative artificial intelligence model and the specified features of the molecule. Generally, the generative artificial intelligence model may be a model trained to generate a conformer based on minimization of an average flow loss between a modeled flow objective and a ground-truth flow objective associated with an integration over a universe of ground-truth conformer rotations.

In some embodiments, prediction engine 124 can generate the conformer based on an iterative process that adjusts the locations of each atom in the molecule at time t based on a predicted vector field

$${v}_{t}^{θ}$$

at timestep t and the coordinates of each atom in the molecule at an earlier timestep t∈[0,1).

In some embodiments, the generative artificial intelligence model is configured to generate the conformer based on a single step from input to output. To do so, the generative artificial intelligence model may have been refined based on a reflow loss calculated from randomly sampled atom coordinates

$${X}_{0}^{′} and {X}_{1}^{′}$$

from a Gaussian distribution and a generated conformer, respectively. The generative artificial intelligence model may also have been refined based on a distillation loss associated with the pairing of

$$( {X}_{0}^{′} , {X}_{1}^{′} ) .$$

In some embodiments, the generative artificial intelligence model comprises a convolutional network in which atom feature information is mixed with a relative distance vector associated the average flow loss.

In some embodiments, the generative artificial intelligence model is further trained to generate the conformer from a noise distribution based on a direct path from the noise distribution to a target distribution associated with the conformer.

At block 430, prediction engine 124 outputs the generated conformer. This conformer can be used for various purposes in drug discovery and development. For example, it can be used in binding affinity studies to evaluate how well the conformer interacts with target proteins or receptors. In drug design, the conformer may assist in lead optimization and computational screening to identify promising drug candidates. Additionally, it may support chemical synthesis planning and formulation development to ensure the drug has the desired physical properties for effective delivery.

By training a generative model to generate a conformer from a two-dimensional graph representation of a molecule based on an average flow loss calculated over a universe of rotations of a conformer, embodiments presented herein generally allow for increased training and inferencing efficiency relative to models trained based on rotations of prior and target data distributions or other techniques in which a loss used in training a model is rotation-specific. At inferencing time, embodiments presented herein may allow for conformer generation using fewer inferencing rounds than generative artificial intelligence models trained using rotation-specific techniques, thus saving computing resources (e.g., power, processing time, memory utilization, etc.) relative to the amount of computing resources used by generative artificial intelligence models trained using rotation-specific techniques. Finally, training a generative artificial intelligence model based on an average flow loss calculated over a universe of rotations of a conformer, embodiments presented herein may allow for increased conformer accuracy and validity relative to conformers generated by models trained using rotation-specific techniques.

### Inference and Training Logic

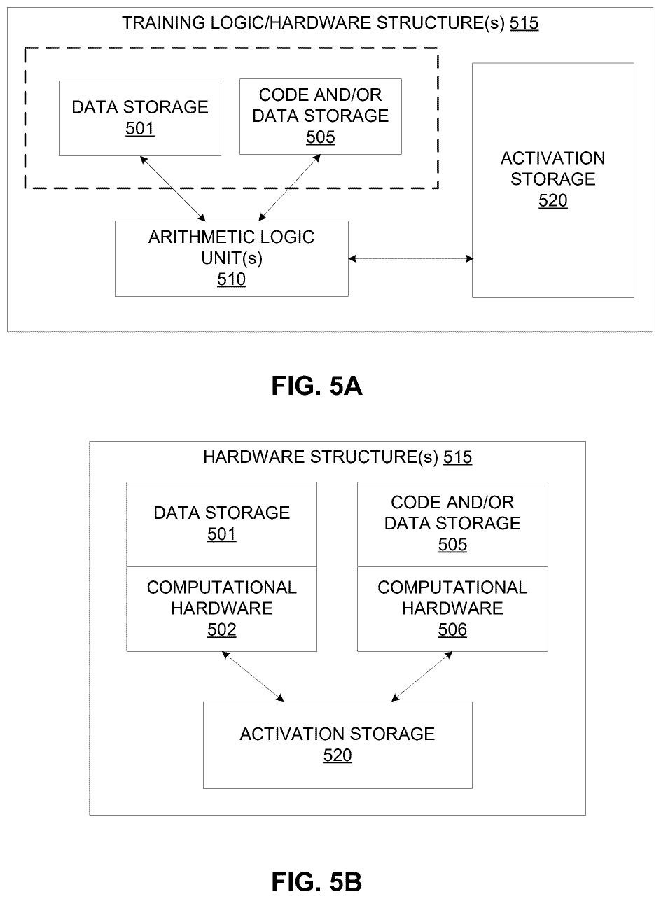

FIG. 5A illustrates inference and/or training logic 515 used to perform inferencing and/or training operations associated with one or more embodiments. Details regarding inference and/or training logic 515 are provided herein in conjunction with at least FIGS. 5A and/or 5B.

In at least one embodiment, inference and/or training logic 515 may include, without limitation, code and/or data storage 501 to store forward and/or output weight and/or input/output data, and/or other parameters to configure neurons or layers of a neural network trained and/or used for inferencing in aspects of one or more embodiments. In at least one embodiment, training logic 515 may include, or be coupled to code and/or data storage 501 to store graph code or other software to control timing and/or order, in which weight and/or other parameter information is to be loaded to configure, logic, including integer and/or floating point units (collectively, arithmetic logic units (ALUs)). In at least one embodiment, code, such as graph code, loads weight or other parameter information into processor ALUs based on an architecture of a neural network to which such code corresponds. In at least one embodiment, code and/or data storage 501 stores weight parameters and/or input/output data of each layer of a neural network trained or used in conjunction with one or more embodiments during forward propagation of input/output data and/or weight parameters during training and/or inferencing using aspects of one or more embodiments. In at least one embodiment, any portion of code and/or data storage 501 may be included with other on-chip or off-chip data storage, including a processor's L1, L2, or L3 cache or system memory.

In at least one embodiment, any portion of code and/or data storage 501 may be internal or external to one or more processors or other hardware logic devices or circuits. In at least one embodiment, code and/or code and/or data storage 501 may be cache memory, dynamic randomly addressable memory (“DRAM”), static randomly addressable memory (“SRAM”), non-volatile memory (e.g., flash memory), or other storage. In at least one embodiment, a choice of whether code and/or code and/or data storage 501 is internal or external to a processor, for example, or comprising DRAM, SRAM, flash or some other storage type may depend on available storage on-chip versus off-chip, latency requirements of training and/or inferencing functions being performed, batch size of data used in inferencing and/or training of a neural network, or some combination of these factors.

In at least one embodiment, inference and/or training logic 515 may include, without limitation, a code and/or data storage 505 to store backward and/or output weight and/or input/output data corresponding to neurons or layers of a neural network trained and/or used for inferencing in aspects of one or more embodiments. In at least one embodiment, code and/or data storage 505 stores weight parameters and/or input/output data of each layer of a neural network trained or used in conjunction with one or more embodiments during backward propagation of input/output data and/or weight parameters during training and/or inferencing using aspects of one or more embodiments. In at least one embodiment, training logic 515 may include, or be coupled to code and/or data storage 505 to store graph code or other software to control timing and/or order, in which weight and/or other parameter information is to be loaded to configure, logic, including integer and/or floating point units (collectively, arithmetic logic units (ALUs)).

In at least one embodiment, code, such as graph code, causes the loading of weight or other parameter information into processor ALUs based on an architecture of a neural network to which such code corresponds. In at least one embodiment, any portion of code and/or data storage 505 may be included with other on-chip or off-chip data storage, including a processor's L1, L2, or L3 cache or system memory. In at least one embodiment, any portion of code and/or data storage 505 may be internal or external to one or more processors or other hardware logic devices or circuits. In at least one embodiment, code and/or data storage 505 may be cache memory, DRAM, SRAM, non-volatile memory (e.g., flash memory), or other storage. In at least one embodiment, a choice of whether code and/or data storage 505 is internal or external to a processor, for example, or comprising DRAM, SRAM, flash memory or some other storage type may depend on available storage on-chip versus off-chip, latency requirements of training and/or inferencing functions being performed, batch size of data used in inferencing and/or training of a neural network, or some combination of these factors.

In at least one embodiment, code and/or data storage 501 and code and/or data storage 505 may be separate storage structures. In at least one embodiment, code and/or data storage 501 and code and/or data storage 505 may be a combined storage structure. In at least one embodiment, code and/or data storage 501 and code and/or data storage 505 may be partially combined and partially separate. In at least one embodiment, any portion of code and/or data storage 501 and code and/or data storage 505 may be included with other on-chip or off-chip data storage, including a processor's L1, L2, or L3 cache or system memory.

In at least one embodiment, inference and/or training logic 515 may include, without limitation, one or more arithmetic logic unit(s) (“ALU(s)”) 510, including integer and/or floating point units, to perform logical and/or mathematical operations based, at least in part on, or indicated by, training and/or inference code (e.g., graph code), a result of which may produce activations (e.g., output values from layers or neurons within a neural network) stored in an activation storage 520 that are functions of input/output and/or weight parameter data stored in code and/or data storage 501 and/or code and/or data storage 505. In at least one embodiment, activations stored in activation storage 520 are generated according to linear algebraic and or matrix-based mathematics performed by ALU(s) 510 in response to performing instructions or other code, wherein weight values stored in code and/or data storage 505 and/or data storage 501 are used as operands along with other values, such as bias values, gradient information, momentum values, or other parameters or hyperparameters, any or all of which may be stored in code and/or data storage 505 or code and/or data storage 501 or another storage on or off-chip.

In at least one embodiment, ALU(s) 510 are included within one or more processors or other hardware logic devices or circuits, whereas in another embodiment, ALU(s) 510 may be external to a processor or other hardware logic device or circuit that uses them (e.g., a co-processor). In at least one embodiment, ALUs 510 may be included within a processor's execution units or otherwise within a bank of ALUs accessible by a processor's execution units either within same processor or distributed between different processors of different types (e.g., central processing units, graphics processing units, fixed function units, etc.). In at least one embodiment, code and/or data storage 501, code and/or data storage 505, and activation storage 520 may share a processor or other hardware logic device or circuit, whereas in another embodiment, they may be in different processors or other hardware logic devices or circuits, or some combination of same and different processors or other hardware logic devices or circuits. In at least one embodiment, any portion of activation storage 520 may be included with other on-chip or off-chip data storage, including a processor's L1, L2, or L3 cache or system memory. Furthermore, inferencing and/or training code may be stored with other code accessible to a processor or other hardware logic or circuit and fetched and/or processed using a processor's fetch, decode, scheduling, execution, retirement and/or other logical circuits.

In at least one embodiment, activation storage 520 may be cache memory, DRAM, SRAM, non-volatile memory (e.g., flash memory), or other storage. In at least one embodiment, activation storage 520 may be completely or partially within or external to one or more processors or other logical circuits. In at least one embodiment, a choice of whether activation storage 520 is internal or external to a processor, for example, or comprising DRAM, SRAM, flash memory or some other storage type may depend on available storage on-chip versus off-chip, latency requirements of training and/or inferencing functions being performed, batch size of data used in inferencing and/or training of a neural network, or some combination of these factors.

In at least one embodiment, inference and/or training logic 515 illustrated in FIG. 5A may be used in conjunction with an application-specific integrated circuit (“ASIC”), such as a TensorFlow® Processing Unit from Google, an inference processing unit (IPU) from Graphcore™, or a Nervana® (e.g., “Lake Crest”) processor from Intel Corp. In at least one embodiment, inference and/or training logic 515 illustrated in FIG. 5A may be used in conjunction with central processing unit (“CPU”) hardware, graphics processing unit (“GPU”) hardware or other hardware, such as field programmable gate arrays (“FPGAs”).

FIG. 5B illustrates inference and/or training logic 515, according to at least one embodiment. In at least one embodiment, inference and/or training logic 515 may include, without limitation, hardware logic in which computational resources are dedicated or otherwise exclusively used in conjunction with weight values or other information corresponding to one or more layers of neurons within a neural network. In at least one embodiment, inference and/or training logic 515 illustrated in FIG. 5B may be used in conjunction with an application-specific integrated circuit (ASIC), such as TensorFlow® Processing Unit from Google, an inference processing unit (IPU) from Graphcore™, or a Nervana® (e.g., “Lake Crest”) processor from Intel Corp. In at least one embodiment, inference and/or training logic 515 illustrated in FIG. 5B may be used in conjunction with central processing unit (CPU) hardware, graphics processing unit (GPU) hardware or other hardware, such as field programmable gate arrays (FPGAs). In at least one embodiment, inference and/or training logic 515 includes, without limitation, code and/or data storage 501 and code and/or data storage 505, which may be used to store code (e.g., graph code), weight values and/or other information, including bias values, gradient information, momentum values, and/or other parameter or hyperparameter information. In at least one embodiment illustrated in FIG. 5B, each of code and/or data storage 501 and code and/or data storage 505 is associated with a dedicated computational resource, such as computational hardware 502 and computational hardware 506, respectively. In at least one embodiment, each of computational hardware 502 and computational hardware 506 comprises one or more ALUs that perform mathematical functions, such as linear algebraic functions, only on information stored in code and/or data storage 501 and code and/or data storage 505, respectively, result of which is stored in activation storage 520.

In at least one embodiment, each of code and/or data storage 501 and 505 and corresponding computational hardware 502 and 506, respectively, correspond to different layers of a neural network, such that resulting activation from one storage/computational pair 501/502 of code and/or data storage 501 and computational hardware 502 is provided as an input to a next storage/computational pair 505/506 of code and/or data storage 505 and computational hardware 506, in order to mirror a conceptual organization of a neural network. In at least one embodiment, each of storage/computational pairs 501/502 and 505/506 may correspond to more than one neural network layer. In at least one embodiment, additional storage/computation pairs (not shown) subsequent to or in parallel with storage/computation pairs 501/502 and 505/506 may be included in inference and/or training logic 515.

### Neural Network Training and Deployment

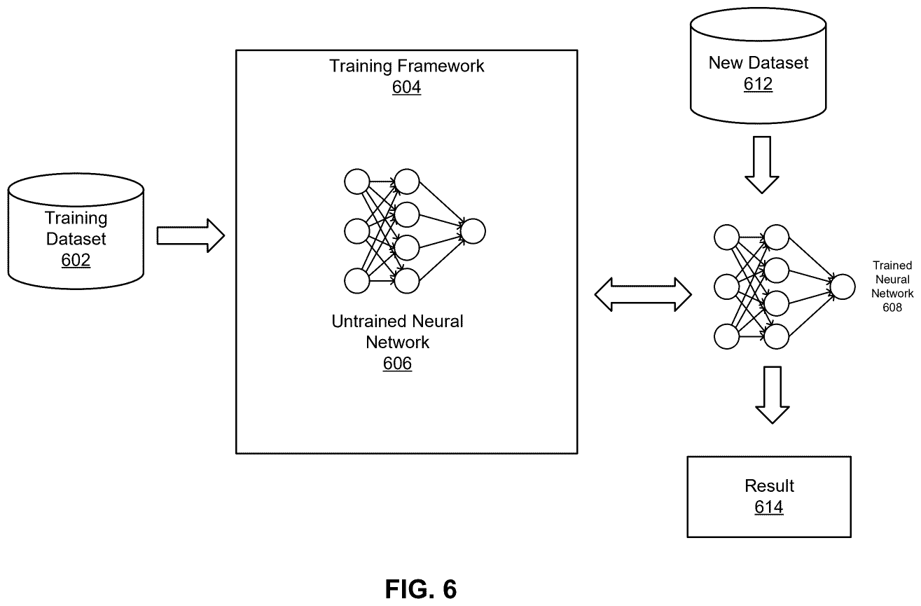

FIG. 6 illustrates training and deployment of a deep neural network, according to at least one embodiment. In at least one embodiment, untrained neural network 606 is trained using a training dataset 602. In at least one embodiment, training framework 604 is a PyTorch framework, whereas in other embodiments, training framework 604 is a TensorFlow, Boost, Caffe, Microsoft Cognitive Toolkit/CNTK, MXNet, Chainer, Keras, Deeplearning4j, or other training framework. In at least one embodiment, training framework 604 trains an untrained neural network 606 and enables it to be trained using processing resources described herein to generate a trained neural network 608. In at least one embodiment, weights may be chosen randomly or by pre-training using a deep belief network. In at least one embodiment, training may be performed in either a supervised, partially supervised, or unsupervised manner.

In at least one embodiment, untrained neural network 606 is trained using supervised learning, wherein training dataset 602 includes an input paired with a desired output for an input, or where training dataset 602 includes input having a known output and an output of neural network 606 is manually graded. In at least one embodiment, untrained neural network 606 is trained in a supervised manner and processes inputs from training dataset 602 and compares resulting outputs against a set of expected or desired outputs. In at least one embodiment, errors are then propagated back through untrained neural network 606. In at least one embodiment, training framework 604 adjusts weights that control untrained neural network 606. In at least one embodiment, training framework 604 includes tools to monitor how well untrained neural network 606 is converging towards a model, such as trained neural network 608, suitable to generating correct answers, such as in result 614, based on input data such as a new dataset 612. In at least one embodiment, training framework 604 trains untrained neural network 606 repeatedly while adjust weights to refine an output of untrained neural network 606 using a loss function and adjustment algorithm, such as stochastic gradient descent. In at least one embodiment, training framework 604 trains untrained neural network 606 until untrained neural network 606 achieves a desired accuracy. In at least one embodiment, trained neural network 608 can then be deployed to implement any number of machine learning operations.

In at least one embodiment, untrained neural network 606 is trained using unsupervised learning, wherein untrained neural network 606 attempts to train itself using unlabeled data. In at least one embodiment, unsupervised learning training dataset 602 will include input data without any associated output data or “ground truth” data. In at least one embodiment, untrained neural network 606 can learn groupings within training dataset 602 and can determine how individual inputs are related to untrained dataset 602. In at least one embodiment, unsupervised training can be used to generate a self-organizing map in trained neural network 608 capable of performing operations useful in reducing dimensionality of new dataset 612. In at least one embodiment, unsupervised training can also be used to perform anomaly detection, which allows identification of data points in new dataset 612 that deviate from normal patterns of new dataset 612.

In at least one embodiment, semi-supervised learning may be used, which is a technique in which in training dataset 602 includes a mix of labeled and unlabeled data. In at least one embodiment, training framework 604 may be used to perform incremental learning, such as through transferred learning techniques. In at least one embodiment, incremental learning enables trained neural network 608 to adapt to new dataset 612 without forgetting knowledge instilled within trained neural network 608 during initial training.

In at least one embodiment, training framework 604 is a framework processed in connection with a software development toolkit such as an OpenVINO (Open Visual Inference and Neural network Optimization) toolkit. In at least one embodiment, an OpenVINO toolkit is a toolkit such as those developed by Intel Corporation of Santa Clara, CA.

In at least one embodiment, OpenVINO is a toolkit for facilitating development of applications, specifically neural network applications, for various tasks and operations, such as human vision emulation, speech recognition, natural language processing, recommendation systems, and/or variations thereof. In at least one embodiment, OpenVINO supports neural networks such as convolutional neural networks (CNNs), recurrent and/or attention-based neural networks, and/or various other neural network models. In at least one embodiment, OpenVINO supports various software libraries such as OpenCV, OpenCL, and/or variations thereof.

In at least one embodiment, OpenVINO supports neural network models for various tasks and operations, such as classification, segmentation, object detection, face recognition, speech recognition, pose estimation (e.g., humans and/or objects), monocular depth estimation, image inpainting, style transfer, action recognition, colorization, and/or variations thereof.

In at least one embodiment, OpenVINO comprises one or more software tools and/or modules for model optimization, also referred to as a model optimizer. In at least one embodiment, a model optimizer is a command line tool that facilitates transitions between training and deployment of neural network models. In at least one embodiment, a model optimizer optimizes neural network models for execution on various devices and/or processing units, such as a GPU, CPU, PPU, GPGPU, and/or variations thereof. In at least one embodiment, a model optimizer generates an internal representation of a model, and optimizes said model to generate an intermediate representation. In at least one embodiment, a model optimizer reduces a number of layers of a model. In at least one embodiment, a model optimizer removes layers of a model that are utilized for training. In at least one embodiment, a model optimizer performs various neural network operations, such as modifying inputs to a model (e.g., resizing inputs to a model), modifying a size of inputs of a model (e.g., modifying a batch size of a model), modifying a model structure (e.g., modifying layers of a model), normalization, standardization, quantization (e.g., converting weights of a model from a first representation, such as floating point, to a second representation, such as integer), and/or variations thereof.

In at least one embodiment, OpenVINO comprises one or more software libraries for inferencing, also referred to as an inference engine. In at least one embodiment, an inference engine is a C++ library, or any suitable programming language library. In at least one embodiment, an inference engine is utilized to infer input data. In at least one embodiment, an inference engine implements various classes to infer input data and generate one or more results. In at least one embodiment, an inference engine implements one or more API functions to process an intermediate representation, set input and/or output formats, and/or execute a model on one or more devices.

In at least one embodiment, OpenVINO provides various abilities for heterogeneous execution of one or more neural network models. In at least one embodiment, heterogeneous execution, or heterogeneous computing, refers to one or more computing processes and/or systems that utilize one or more types of processors and/or cores. In at least one embodiment, OpenVINO provides various software functions to execute a program on one or more devices. In at least one embodiment, OpenVINO provides various software functions to execute a program and/or portions of a program on different devices. In at least one embodiment, OpenVINO provides various software functions to, for example, run a first portion of code on a CPU and a second portion of code on a GPU and/or FPGA. In at least one embodiment, Open VINO provides various software functions to execute one or more layers of a neural network on one or more devices (e.g., a first set of layers on a first device, such as a GPU, and a second set of layers on a second device, such as a CPU).

In at least one embodiment, OpenVINO includes various functionality similar to functionalities associated with a CUDA programming model, such as various neural network model operations associated with frameworks such as TensorFlow, PyTorch, and/or variations thereof. In at least one embodiment, one or more CUDA programming model operations are performed using OpenVINO. In at least one embodiment, various systems, methods, and/or techniques described herein are implemented using OpenVINO.

Other variations are within spirit of present disclosure. Thus, while disclosed techniques are susceptible to various modifications and alternative constructions, certain illustrated embodiments thereof are shown in drawings and have been described herein in detail. It should be understood, however, that there is no intention to limit disclosure to specific form or forms disclosed, but on contrary, intention is to cover all modifications, alternative constructions, and equivalents falling within spirit and scope of disclosure, as defined in appended claims.

Use of terms “a” and “an” and “the” and similar referents in context of describing disclosed embodiments (especially in context of following claims) are to be construed to cover both singular and plural, unless otherwise indicated herein or clearly contradicted by context, and not as a definition of a term. Terms “comprising,” “having,” “including,” and “containing” are to be construed as open-ended terms (meaning “including, but not limited to,”) unless otherwise noted. “Connected,” when unmodified and referring to physical connections, is to be construed as partly or wholly contained within, attached to, or joined together, even if there is something intervening. Recitation of ranges of values herein are merely intended to serve as a shorthand method of referring individually to each separate value falling within range, unless otherwise indicated herein and each separate value is incorporated into specification as if it were individually recited herein. In at least one embodiment, use of term “set” (e.g., “a set of items”) or “subset” unless otherwise noted or contradicted by context, is to be construed as a nonempty collection comprising one or more members. Further, unless otherwise noted or contradicted by context, term “subset” of a corresponding set does not necessarily denote a proper subset of corresponding set, but subset and corresponding set may be equal.

Conjunctive language, such as phrases of form “at least one of A, B, and C,” or “at least one of A, B and C,” unless specifically stated otherwise or otherwise clearly contradicted by context, is otherwise understood with context as used in general to present that an item, term, etc., may be either A or B or C, or any nonempty subset of set of A and B and C. For instance, in illustrative example of a set having three members, conjunctive phrases “at least one of A, B, and C” and “at least one of A, B and C” refer to any of following sets: {A}, {B}, {C}, {A, B}, {A, C}, {B, C}, {A, B, C}. Thus, such conjunctive language is not generally intended to imply that certain embodiments require at least one of A, at least one of B and at least one of C each to be present. In addition, unless otherwise noted or contradicted by context, term “plurality” indicates a state of being plural (e.g., “a plurality of items” indicates multiple items). In at least one embodiment, number of items in a plurality is at least two, but can be more when so indicated either explicitly or by context. Further, unless stated otherwise or otherwise clear from context, phrase “based on” means “based at least in part on” and not “based solely on.”

Operations of processes described herein can be performed in any suitable order unless otherwise indicated herein or otherwise clearly contradicted by context. In at least one embodiment, a process such as those processes described herein (or variations and/or combinations thereof) is performed under control of one or more computer systems configured with executable instructions and is implemented as code (e.g., executable instructions, one or more computer programs or one or more applications) executing collectively on one or more processors, by hardware or combinations thereof. In at least one embodiment, code is stored on a computer-readable storage medium, for example, in form of a computer program comprising a plurality of instructions executable by one or more processors. In at least one embodiment, a computer-readable storage medium is a non-transitory computer-readable storage medium that excludes transitory signals (e.g., a propagating transient electric or electromagnetic transmission) but includes non-transitory data storage circuitry (e.g., buffers, cache, and queues) within transceivers of transitory signals. In at least one embodiment, code (e.g., executable code or source code) is stored on a set of one or more non-transitory computer-readable storage media having stored thereon executable instructions (or other memory to store executable instructions) that, when executed (i.e., as a result of being executed) by one or more processors of a computer system, cause computer system to perform operations described herein. In at least one embodiment, set of non-transitory computer-readable storage media comprises multiple non-transitory computer-readable storage media and one or more of individual non-transitory storage media of multiple non-transitory computer-readable storage media lack all of code while multiple non-transitory computer-readable storage media collectively store all of code. In at least one embodiment, executable instructions are executed such that different instructions are executed by different processors—for example, a non-transitory computer-readable storage medium store instructions and a main central processing unit (“CPU”) executes some of instructions while a graphics processing unit (“GPU”) executes other instructions. In at least one embodiment, different components of a computer system have separate processors and different processors execute different subsets of instructions.

In at least one embodiment, an arithmetic logic unit is a set of combinational logic circuitry that takes one or more inputs to produce a result. In at least one embodiment, an arithmetic logic unit is used by a processor to implement mathematical operation such as addition, subtraction, or multiplication. In at least one embodiment, an arithmetic logic unit is used to implement logical operations such as logical AND/OR or XOR. In at least one embodiment, an arithmetic logic unit is stateless, and made from physical switching components such as semiconductor transistors arranged to form logical gates. In at least one embodiment, an arithmetic logic unit may operate internally as a stateful logic circuit with an associated clock. In at least one embodiment, an arithmetic logic unit may be constructed as an asynchronous logic circuit with an internal state not maintained in an associated register set. In at least one embodiment, an arithmetic logic unit is used by a processor to combine operands stored in one or more registers of the processor and produce an output that can be stored by the processor in another register or a memory location.

In at least one embodiment, as a result of processing an instruction retrieved by the processor, the processor presents one or more inputs or operands to an arithmetic logic unit, causing the arithmetic logic unit to produce a result based at least in part on an instruction code provided to inputs of the arithmetic logic unit. In at least one embodiment, the instruction codes provided by the processor to the ALU are based at least in part on the instruction executed by the processor. In at least one embodiment combinational logic in the ALU processes the inputs and produces an output which is placed on a bus within the processor. In at least one embodiment, the processor selects a destination register, memory location, output device, or output storage location on the output bus so that clocking the processor causes the results produced by the ALU to be sent to the desired location.

In the scope of this application, the term arithmetic logic unit, or ALU, is used to refer to any computational logic circuit that processes operands to produce a result. For example, in the present document, the term ALU can refer to a floating point unit, a DSP, a tensor core, a shader core, a coprocessor, or a CPU.

Accordingly, in at least one embodiment, computer systems are configured to implement one or more services that singly or collectively perform operations of processes described herein and such computer systems are configured with applicable hardware and/or software that enable performance of operations. Further, a computer system that implements at least one embodiment of present disclosure is a single device and, in another embodiment, is a distributed computer system comprising multiple devices that operate differently such that distributed computer system performs operations described herein and such that a single device does not perform all operations.

Use of any and all examples, or exemplary language (e.g., “such as”) provided herein, is intended merely to better illuminate embodiments of disclosure and does not pose a limitation on scope of disclosure unless otherwise claimed. No language in specification should be construed as indicating any non-claimed element as essential to practice of disclosure.

All references, including publications, patent applications, and patents, cited herein are hereby incorporated by reference to same extent as if each reference were individually and specifically indicated to be incorporated by reference and were set forth in its entirety herein.

In description and claims, terms “coupled” and “connected,” along with their derivatives, may be used. It should be understood that these terms may be not intended as synonyms for each other. Rather, in particular examples, “connected” or “coupled” may be used to indicate that two or more elements are in direct or indirect physical or electrical contact with each other. “Coupled” may also mean that two or more elements are not in direct contact with each other, but yet still co-operate or interact with each other.

Unless specifically stated otherwise, it may be appreciated that throughout specification terms such as “processing,” “computing,” “calculating,” “determining,” or like, refer to action and/or processes of a computer or computing system, or similar electronic computing device, that manipulate and/or transform data represented as physical, such as electronic, quantities within computing system's registers and/or memories into other data similarly represented as physical quantities within computing system's memories, registers or other such information storage, transmission or display devices.

In a similar manner, term “processor” may refer to any device or portion of a device that processes electronic data from registers and/or memory and transform that electronic data into other electronic data that may be stored in registers and/or memory. As non-limiting examples, “processor” may be a CPU or a GPU. A “computing platform” may comprise one or more processors. As used herein, “software” processes may include, for example, software and/or hardware entities that perform work over time, such as tasks, threads, and intelligent agents. Also, each process may refer to multiple processes, for carrying out instructions in sequence or in parallel, continuously or intermittently. In at least one embodiment, terms “system” and “method” are used herein interchangeably insofar as system may embody one or more methods and methods may be considered a system.

In present document, references may be made to obtaining, acquiring, receiving, or inputting analog or digital data into a subsystem, computer system, or computer-implemented machine. In at least one embodiment, process of obtaining, acquiring, receiving, or inputting analog and digital data can be accomplished in a variety of ways such as by receiving data as a parameter of a function call or a call to an application programming interface. In at least one embodiment, processes of obtaining, acquiring, receiving, or inputting analog or digital data can be accomplished by transferring data via a serial or parallel interface. In at least one embodiment, processes of obtaining, acquiring, receiving, or inputting analog or digital data can be accomplished by transferring data via a computer network from providing entity to acquiring entity. In at least one embodiment, references may also be made to providing, outputting, transmitting, sending, or presenting analog or digital data. In various examples, processes of providing, outputting, transmitting, sending, or presenting analog or digital data can be accomplished by transferring data as an input or output parameter of a function call, a parameter of an application programming interface or interprocess communication mechanism.

Although descriptions herein set forth example implementations of described techniques, other architectures may be used to implement described functionality, and are intended to be within scope of this disclosure. Furthermore, although specific distributions of responsibilities may be defined above for purposes of description, various functions and responsibilities might be distributed and divided in different ways, depending on circumstances.

### EXAMPLE CLAUSES

Implementation details of various embodiments of the present disclosure are described in the following numbered clauses

1. In some embodiments, a method comprises modeling a flow objective over a universe of conformers for a molecule having a defined set of atoms and bonds between atoms, the flow objective being modeled based on an integration over a universe of ground-truth conformer rotations; calculating an average flow loss based on a difference between the modeled flow objective and a ground-truth flow objective associated with the integration over the universe of ground-truth conformer rotations; training a generative model to generate a conformer given an input of atoms and bonds of a target molecule, the training being based at least on minimizing the average flow loss; and deploying the trained generative model.

2. The method of clause 1, further comprising refining the generative model based on a reflow loss calculated based on a position of an atom in the molecule sampled from a noise distribution and a position of the atom in a denoised version of the molecule generated by the generative model.

3. The method of any of clauses 1 or 2, further comprising refining the generative model based on a distillation loss based on a relationship between a position of an atom in the molecule sampled from a noise distribution and a position of the atom in a denoised version of the molecule generated by the generative model.

4. The method of any of clauses 1 through 3, wherein the integration over the universe of ground-truth conformer rotations is calculated based on an average atom location over the universe of rotations for an atom in the molecule.

5. The method of any of clauses 1 through 4, wherein the generative model is trained to generate the conformer based on a direct line from an initial point and a fixed point in a single time step.

6. The method of any of clauses 1 through 5, wherein the modeled flow objective comprises a learned vector field associated with a generated conformer and the ground-truth flow objective comprises a ground-truth vector field associated with a ground-truth conformer.

7. The method of clause 6, wherein the ground-truth vector field comprises a vector field calculated based on a normalized summation of integrals over each rotation of the ground-truth conformer in the universe of ground-truth conformer rotations.

8. The method of clause 7, wherein the normalized summation of integrals over each rotation of the ground-truth conformer in the universe of ground-truth conformer rotations is calculated based on a partial derivative of a partition function over the universe of ground-truth conformer rotations and a location of an intermediate particle.

9. The method of any of clauses 1 through 8, wherein the universe of ground-truth conformer rotations comprise an ensemble of conformers for the molecule, each conformer in the ensemble of conformers corresponding to the molecule at a local minimum in a conformational energy landscape.

10. In some embodiments, a processor-implemented method comprises receiving a request to generate a conformer using a generative artificial intelligence model, the request specifying features of a molecule associated with the conformer; generating the conformer based on the generative artificial intelligence model and the specified features of the molecule, the generative artificial intelligence model comprising a model trained to generate a conformer based on minimization of an average flow loss between a modeled flow objective and a ground-truth flow objective associated with an integration over a universe of ground-truth conformer rotations; and outputting the generated conformer.

11. The method of clause 10, wherein the features of the molecule associated with the conformer comprise atom type, bonds between atoms in the molecule, and a type of each bond between atoms in the molecule.

12. The method of any of clauses 10 or 11, wherein the generative artificial intelligence model is configured to generate the conformer based on a single step from input to output.

13. The method of any of clauses 10 through 12, wherein the generative artificial intelligence model comprises a convolutional network in which atom feature information is mixed with a relative distance vector associated the average flow loss.

14. The method of any of clauses 10 through 13, wherein the generative artificial intelligence model is further trained to generate the conformer from a noise distribution based on a direct path from the noise distribution to a target distribution associated with the conformer.

15. A processing system, comprising: at least one memory having executable instructions stored thereon; and one or more processors configured to execute the operations of any of clauses 1 through 14.

16. A processing system, comprising means for performing the operations of any of clauses 1 through 14.

17. A non-transitory computer readable medium having executable instructions stored thereon which, when executed by one or more processors, performs the operations of any of clauses 1 through 14.

Furthermore, although subject matter has been described in language specific to structural features and/or methodological acts, it is to be understood that subject matter claimed in appended claims is not necessarily limited to specific features or acts described. Rather, specific features and acts are disclosed as exemplary forms of implementing the claims.

What is claimed is:

1. A processor-implemented method, comprising:
modeling a flow objective over a universe of conformers for a molecule having a defined set of atoms and bonds between atoms, the flow objective being modeled based on an integration over a universe of ground-truth conformer rotations;
calculating an average flow loss based on a difference between the modeled flow objective and a ground-truth flow objective associated with the integration over the universe of ground-truth conformer rotations;
training a generative model to generate a conformer given an input of atoms and bonds of a target molecule, the training being based at least on minimizing the average flow loss; and
deploying the trained generative model.

2. The method of claim 1, further comprising refining the generative model based on a reflow loss calculated based on a position of an atom in the molecule sampled from a noise distribution and a position of the atom in a denoised version of the molecule generated by the generative model.

3. The method of claim 1, further comprising refining the generative model based on a distillation loss based on a relationship between a position of an atom in the molecule sampled from a noise distribution and a position of the atom in a denoised version of the molecule generated by the generative model.

4. The method of claim 1, wherein the integration over the universe of ground-truth conformer rotations is calculated based on an average atom location over the universe of rotations for an atom in the molecule.

5. The method of claim 1, wherein the generative model is trained to generate the conformer based on a direct line from an initial point and a fixed point in a single time step.

6. The method of claim 1, wherein the modeled flow objective comprises a learned vector field associated with a generated conformer and the ground-truth flow objective comprises a ground-truth vector field associated with a ground-truth conformer.

7. The method of claim 6, wherein the ground-truth vector field comprises a vector field calculated based on a normalized summation of integrals over each rotation of the ground-truth conformer in the universe of ground-truth conformer rotations.

8. The method of claim 7, wherein the normalized summation of integrals over each rotation of the ground-truth conformer in the universe of ground-truth conformer rotations is calculated based on a partial derivative of a partition function over the universe of ground-truth conformer rotations and a location of an intermediate particle.

9. The method of claim 1, wherein the universe of ground-truth conformer rotations comprise an ensemble of conformers for the molecule, each conformer in the ensemble of conformers corresponding to the molecule at a local minimum in a conformational energy landscape.

10. A processor-implemented method, comprising:
receiving a request to generate a conformer using a generative artificial intelligence model, the request specifying features of a molecule associated with the conformer;
generating the conformer based on the generative artificial intelligence model and the specified features of the molecule, the generative artificial intelligence model comprising a model trained to generate the conformer based on minimization of an average flow loss between a modeled flow objective for the molecule and a ground-truth flow objective associated with an integration over a universe of ground-truth conformer rotations; and
outputting the generated conformer.

11. The method of claim 10, wherein the features of the molecule associated with the conformer comprise atom type, bonds between atoms in the molecule, and a type of each bond between atoms in the molecule.

12. The method of claim 10, wherein the generative artificial intelligence model is configured to generate the conformer based on a single step from input to output.

13. The method of claim 10, wherein the generative artificial intelligence model comprises a convolutional network in which atom feature information is mixed with a relative distance vector associated the average flow loss.

14. The method of claim 10, wherein the generative artificial intelligence model is further trained to generate the conformer from a noise distribution based on a direct path from the noise distribution to a target distribution associated with the conformer.

15. A processing system, comprising:
at least one memory having executable instructions stored thereon; and
one or more processors configured to execute the executable instructions to cause the processing system to:
receive a request to generate a three-dimensional conformer of a molecule using a generative artificial intelligence model, the request including a two-dimensional graph representation of the molecule;
generate the three-dimensional conformer based on the generative artificial intelligence model, the generative artificial intelligence model trained to generate conformers by minimizing of an average flow loss between a modeled flow objective for the molecule and a ground-truth flow objective associated with an integration over a universe of ground-truth conformer rotations; and
output the generated three-dimensional conformer.

16. The processing system of claim 15, wherein the two-dimensional graph representation of the molecule defines atom type, bonds between atoms in the molecule, and a type of each bond between atoms in the molecule.

17. The processing system of claim 15, wherein the generative artificial intelligence model is configured to generate the conformer based on a single step from input to output.

18. The processing system of claim 15, wherein the generative artificial intelligence model comprises a convolutional network in which atom feature information is mixed with a relative distance vector associated the average flow loss.

19. The processing system of claim 15, wherein the generative artificial intelligence model is further trained to generate the conformer from a noise distribution based on a direct path from the noise distribution to a target distribution associated with the conformer.

20. The processing system of claim 15, wherein the system is comprised in at least one of:
a system for performing simulation operations;
a system for performing digital twin operations;
a system for performing collaborative content creation for 3D assets;
a system for performing one or more deep learning operations;
a system implemented using an edge device;
a system for generating or presenting at least one of virtual reality content, augmented reality content, or mixed reality content;
a system implemented using a robot;
a system for performing one or more conversational AI operations;
a system implemented using one or more large language models (LLMs);
a system implementing one or more vision language models (VLMs);
a system implementing one or more multi modal language models;
a system for generating synthetic data;
a system for performing one or more generative AI operations;
a system incorporating one or more virtual machines (VMs);
a system implemented at least partially in a data center; or
a system implemented at least partially using cloud computing resources.
